(19) United States
(12) Patent Application Publication
(10) Pub. No.: US 2013/0137759 A1
(43) Pub. Date: May 30, 2013

(54) COMPOSITIONS AND METHODS FOR TREATING PARASITIC DISEASE

(71) Applicant: Pamela J. Weathers, Slow, MA (US)

(72) Inventor: Pamela J. Weathers, Slow, MA (US)

(73) Assignee: Worcester Polytechnic Institute, Worcester, MA (US)

(21) Appl. No.: 13/582,917

(22) PCT Filed: Mar. 4, 2011
(86) PCT No.: PCT/US2011/027256

§ 371 Date: Dec. 17, 2012

Related U.S. Application Data
(60) Provisional application No. 61/311,075, filed on Mar. 5, 2010.

(51) Int. Cl. A61K 36/282; A61K 45/06; A61K 31/366

(52) U.S. Cl. CPC A61K 36/282; A61K 31/366; A61K 45/06
USPC 514/450

(57) ABSTRACT

The present invention features compositions and methods for treatment of parasitic diseases and cancer. The compositions include Artemisia annua tissue and a pharmaceutically acceptable carrier. The methods provide an efficient delivery system for artemisinin and related compounds.

### CROSS-REFERENCE TO RELATED APPLICATIONS

This application claims the benefit of the filing date of U.S. Provisional Application No. 61/311,075, which was filed on Mar. 5, 2010. For the purpose of any U.S. application that may claim the benefit of U.S. Provisional Application No. 61/311,075, the contents of that earlier filed application are hereby incorporated by reference in their entirety.

### BACKGROUND

In low income and developing nations, malaria is the fifth most prevalent infectious disease and the tenth overall cause of death, and is projected to remain at that level until at least 2030 (Mathers et al. 2006, PLoS Med 3: e442). The World Health Organization (WHO) estimates that more than 380 million cases of malaria occur each year and account for more than 1 million deaths especially in developing countries (Rathore et al., 2005, Expert Opin Investig Drugs 14:871-883).

### SUMMARY OF THE INVENTION

The present invention is based, in part, on studies of the plant Artemisia annua and on evidence that tissue from this plant can be formulated into pharmaceutical compositions and used to treat a variety of unwanted conditions, including infectious disease (e.g., parasitic infections), inflammation, and neoplasms. While the compositions of the invention are not so limited, the formulations can be straightforward in their form and content. For example, they can include or consist of dried plant tissue and a pharmaceutically acceptable carrier, such as a capsule or other vessel that binds or contains the plant tissue and that allows for oral administration to a subject (e.g., a human patient). Carriers that shield the plant material in the mouth may be preferred, as the material is bitter.

While Artemisia annua is currently the only known source of artemisinin, the present compositions can be made with any plant tissue that naturally contains sufficient artemisinin or that is bred or engineered to contain sufficient artemisinin and/or beneficial levels of other compounds, such as plant flavenoids. The plant tissue used in the present compositions can be harvested from one or more parts of the plant, including one or more of the roots, shoots, stems, leaves, floral buds, and flowers. Further, the tissue can be harvested at a time when artemesinin levels are at their highest or approaching their highest levels. For example, the tissue can be harvested when the plant is budding or just prior to full flower opening. More quantitatively, the tissue can be harvested when artemisinin constitutes between about 0.1-5.0% of the dry weight of the tissue (e.g., about 0.5-3.0% dry weight). The production methods of the invention can include a step in which artemesinin levels are assessed prior to harvesting the tissue. Similarly, the production methods can include a step in which another plant compound, such as a flavenoid, can be measured as well. While the invention is not so limited, there may be a synergistic effect between artemesinin and plant flavenoids.

As noted above, the plant tissue can be manipulated (e.g., compacted or shredded). Further, the compositions of the invention can include plant material and other substances. For example, the plant material can be formulated in a container (e.g., a capsule) with purified or synthesized compounds, including purified or synthesized artemisinin and/or a plant flavenoid. However, it is to be understood that the terms “plant tissue” or “Artemisia annua tissue” do not mean pure or substantially purified preparations of chemical compounds.

Following harvest, and in any of the compositions of the present invention, the plant tissue can be simply compacted or it can be disrupted in some way before being incorporated with a carrier. For example, the tissue can be shredded, cut, granulated, pulverised, ground, powdered, or the like. Following harvest, and in any of the compositions of the present invention, the plant tissue can be dried, and it may be dried before or after it is disrupted by shredding or any of the other means just described. The extent to which the plant material is dried can vary. In some embodiments, it will be thoroughly dried, and the present pharmaceutical compositions can further include, or can be packaged with, a dessicant. In the production methods, the plant tissue can be dried naturally (e.g., simply air dried) or dried with the assistance of applied heat or air.

A variety of pharmaceutically acceptable carriers can be used, so long as they have no significant detrimental effect upon ingestion (e.g., little or no toxicity). For example, the carrier can be, or can include, naturally occurring or synthetic materials, including those known, and used in the art of medicinal chemistry and pharmacy. For example, the carrier can be, or can include, a lipid-based or polymer-based colloid. The carrier can surround the Artemisia annua tissue, such that the tissue essentially constitutes an inner layer of the composition; the carrier can be interspersed, uniformly or non-uniformly, the Artemisia annua tissue; or the composition may include carrier material that both surrounds the Artemisia annua tissue and is interspersed with the tissue. Carrier materials in the surround and in the core may be the same or different.

In one embodiment, the carrier material can be a colloid formulated as a liposome, a hydrogel, a micropaticle, a nanoparticle, or a block copolymer micelle. As noted, the carrier material can form a capsule, and that material may be a polymer-based colloid.

With respect to dosage, the amount of plant material incorporated in a unit dosage can vary, and the amount may be modified depending on the subject to be treated (e.g., depending on whether the subject is an adult or child). As the pharmaceutical compositions can be configured for oral administration, and as children may have more difficulty swallowing the formulation (whether presented as a capsule, tablet, or other “pill” form), compositions prepared for administration to children may include less plant material and/or the material may be divided among unit dosage forms.

Any of the compositions described herein can be formulated such that the tissue is in a unit dosage form of about 0.1 grams to about 5.0 grams (e.g., about 0.1, 0.2, 0.3, 1.0, 1.5, 2.0, 2.5, 3.0, 3.5, or 4.0 grams; with ranges being selected from between any lower and higher level (e.g., about 0.5-1.0 gram, 1.0-2.0 grams, or about 2.5-3.0 grams).

The dosage may also be expressed as the amount that gives rise to a circulating blood or plasma level. For example, a unit dosage form can include an amount of Artemisia annua sufficient, when administered to a subject, to result in a circulating concentration of artemesinin in the subject of more than 0.2 mg/L (e.g., about 0.3 mg/L to about 1.0 mg/L (e.g., about 0.4, 0.5, 0.6, 0.7, or 0.8 mg/L)).

The pharmaceutical compositions described herein can include, in addition to the Artemisia annua tissue, a second therapeutic agent or a compound that enhances the efficacy of artemisinin and/or the Artemisia annua tissue. For example, the pharmaceutical compositions can include an agent for treating an infectious disease (e.g., an anti-parasitic agent, such as an anti-malarial agent). Useful anti-malarial agents are known in the art and include lumefantrine, mefloquine, amodiaquine and sulfadoxine/pyrimethamine. Where the subject to be treated is suffering from inflammation, the pharmaceutical compositions can include an anti-inflammatory agent. Where the subject to be treated is suffering from cancer, the pharmaceutical compositions can include a chemotherapeutic agent.

The methods of the present invention include those for treating a subject who has an infectious disease (e.g., a parasitic disease), an inflammatory disease, or cancer. The methods include a step of administering to the subject an effective amount of a pharmaceutical composition as described herein (e.g., a composition comprising Artemisia annua tissue and a pharmaceutically acceptable carrier). In any of the methods, one can include a step of identifying a subject amenable to treatment (by, for example, conducting a diagnostic test for the suspected condition). The subject can be human, but we expect the present methods to be applied in veterinary contexts as well.

In addition to malaria (e.g., falciparum malaria or vivax malaria), the parasitic disease schistosomiasis can also be treated.

While we have expressed the invention, in part, in terms of methods of treatment, any of these aspects of the invention can be expressed in terms of “use.” For example, the invention features use of a composition described herein the preparation of a medicament and use of a composition described herein in the preparation of a medicament for the treatment of, for example, infectious disease, an inflammatory disease, or cancer

The details of one or more embodiments of the invention are set forth in the drawings and the description below. Other features, objects, and advantages of the invention will be apparent from the description and drawings, and from the claims.

### DETAILED DESCRIPTION

The present invention is based, in part, on our studies of artemesinin production in the herbaceous plant, Artemisia annua. We asked whether mice that had been fed the leaves of A. annua would have detectable circulating levels of artemesinin. We discovered that oral administration of A. annua leaves produced levels of artemesinin in the bloodstream that were comparable to those observed in mice that had received purified artemesinin. Surprisingly, we found that the plant material provided a much more efficient transfer of artemesinin into the bloodstream than did the purified artemesinin. Accordingly, the invention features methods and compositions for treating a subject who has a parasitic infection.

Artemisia annua produces the sesquiterpene lactone, artemisinin, a potent antimalarial drug that is also effective in treating other parasitic diseases, some viral infections and various neoplasms. Artemisinin is also an allelopathic herbicide that can inhibit the growth of other plants. Unfortunately, the compound is in short supply and thus, studies on its production in the plant are of interest as are low cost methods for drug delivery. Here we describe our recent studies on artemisinin production in A. annua during development of the plant as it moves from the vegetative to reproductive stage (flower budding and fall flower formation), in response to sugars, and in concert with the production of the ROS, hydrogen peroxide. We also provide new data from animal experiments that measured the potential of using the dried plant directly as a therapeutic. Together these results provide a synopsis of a more global view of regulation of artemisinin biosynthesis in A. annua than previously available. We further suggest an alternative low cost method of drug delivery to treat malaria and other neglected tropical diseases. Accordingly, the present invention features pharmaceutical formulations that include artemisinin in planta and methods of treating patients who are suffering from malaria, other parasitic disease, viral infection, and neoplasms. The pathogens with which a patient can be infected include Pneumocystis carinii and Toxoplasma gondii. The patient may be suffering from a parasitic tropical disease, including schistosomiasis (Utzinger et al., 2001, Curr Medicin Chem 8:1841-1860), leishmaniasis (Sen et al., 2007, J Med Microbiol 56:1213-1218), Chagas disease, and African sleeping sickness (Mishina et al., 2007, Antimicrob Agents Chemother 51:1852-1854).

Although total chemical synthesis of artemisinin has been achieved, it is not cost effective (Haynes, 2006, Curr Top Med Chem 6:509-537). Current technology for artemisinin production is based on cultivated A. annua with best cultivars giving yields of artemisinin of ca. 1.5% of dry plant material and 70 kg/ha (Kumar et al., 2004, Indust Crops Products 19:77-90). Artemisinin is solvent-extracted from plant material, crystallized, and typically used for semi-synthesis of artemisinin derivatives (Haynes, 2006, Curr Top Med Chem 6:509-537). While A. annua is relatively easy to grow in temperate climates, low yields of artemisinin result in relatively high costs for isolation and purification of the useful chemical. The relatively long agricultural timeframe also results in wide swings in supply and price as demand changes. Although scientists at University of York, UK and elsewhere are breeding cultivars of A. annua for higher trichome densities and, thus, artemisinin production (Grove et al., 2007, Eur J Trop Med Internal Health 12 (Supplement 1): 68), and transgenic production schemes are in progress (Arsenault et al., 2008, Curr Medicin Chem 15:2886-2896), there is still a world-wide shortage of the drug just tor treating malaria let alone any other diseases against which artemisinin holds such promise (deRidder et al., 2008, J Ethnopharmacol 120:302-314). Clearly more low cost production and delivery of artemisinin as WHO recommended Artemisinin Combination Therapy (ACT) are needed.

Considering that this drug must be produced cheaply in much greater quantities than currently available, we summarize here our recent work to better explain artemisinin production in planta. We also provide preliminary data from feeding studies with mice that suggest a new approach for drug delivery could be implemented using encapsulated dried leaves of the plant and an ACT counterpart to minimize the emergence of resistance. This same drug delivery approach, without the ACT, could also be used to treat other neglected tropical diseases that are apparently susceptible to artemisinin such schistosomiasis, Chagas disease, and African sleeping sickness.

Artemisinin Biosynthesis:

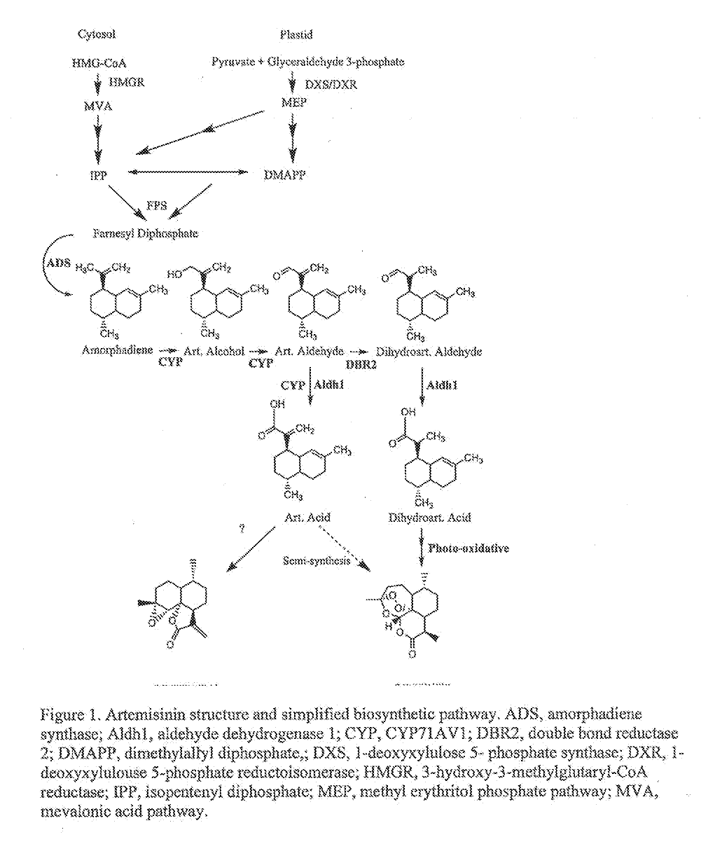

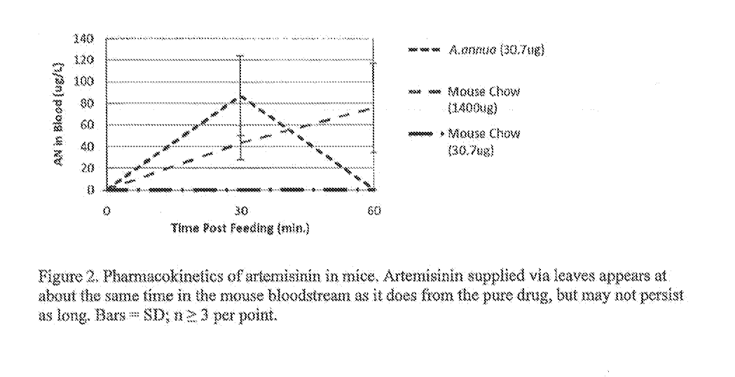

Through recent work from several groups, the biosynthesis of the sesquiterpene, artemisinin, is almost completely resolved (FIG. 1). Artemisinin derives from the condensation of three 5-carbon isoprenoid molecules that originate from both the plastid and cytosol (Towler and Weathers, 2007, Plant Cell Rep 26:2129-2136). These two arms of the terpenoid pathway up to farnesyl diphosphate are regulated in large part by 1-deoxyxylulose 5-phosphate synthase (DXS), and 1-deoxyxylulouse 5-phosphate reductoisomerase (DXR) or 3-hydroxy-3-methylglutaryl-CoA reductase (HMGR) respectively, finally leading to the production of farnesyl diphosphate via farnesyl diphosphate synthase (FPS). Farnesyl diphosphate is then converted to amorpha-4,11-diene via amorphadiene synthase (ADS; Bouwmeester et al 1999, Phytochem 52:843-854, Picaud 2005, Archives of Biochemistry and Biophysics. 436:215-226). The majority of data support the role of dihydroartemisinic acid (DHAA) as a late intermediate in artemisinin biosynthesis (FIG. 1; Zhang et al. 2008, J Biol Chem 283:21501-8). DHAA is formed via artemisinic aldehyde by the action of the cytochrome P450, CYP71AV1 (Teoh et al. 2006, FEBS Lett 580:1411-1416, Ro et al. 2006), DBR2 (Zhang et al. 2008, J Biol Chem 283:21501-8) and probably ALDH1 (Teoh et al. 2009, Botany 87:635-642). DHAA is believed to be converted to artemisinin (AN) non-enzymatically (Covello 2008, Phytochemistry. 69:2881-2885). The pathway also branches at artemisinic aldehyde to give artemisinic acid (AA) by the action of CYP71AV1 and/or ALDH1, and arteannuin B (AB), possibly nonenzymatically. The genes encoding ADS, CYP71AV1, DBR2 and ALDH1 are all preferentially expressed in glandular trichomes.

Artemisinin Production and Trichomes are Intimately Related:

Artemisinin is produced in glandular trichomes that are found on leaves, floral buds, and flowers (Ferreira and Janick, 1995, Int. J. Plant Sci 156:807-815; Tellez et al., 1999, Photochem 52:1035-1040). During vegetative growth of A. annua plants, trichome numbers increase on the leaf surface, but when leaf expansion halts, the numbers begin to decline, possibly a result of their collapse (Lommen et al., 2006, Planta Medica 72:336-45). AN increased with trichome numbers, but in some cases AN levels continue to rise even after trichome populations begin collapsing; this was attributed to maturation effects within the trichome (Lommen et al. 2006, Planta Medica 72:336-45).

AN content can vary widely among different cultivars or ecotypes of A. annua (Waallart 2000, Planta Medica 66:57-62), and to the time of harvest, light intensity, and developmental stage (Ferreira and Janick 1995, Int. J. Plant Sci 156:807-815). AN levels reach their peak either just before or at anthesis (Acton et al 1985, Planta Medica 51:441-442, Woerdenbag 1993, Plant Cell Tiss Organ Cult 32:247-257), yet transgenic plants with the flower promoting factor 1 (Fpf1) flowered earlier, but did not produce more AN (Wang et al. 2004, Planta Medica 70:347-52). Thus, other factors linked to flowering are likely more involved in AN increases.

Little is known about how artemisinin and its metabolites are affected throughout plant development and in relation to trichomes. Artemisinin transcripts, metabolite levels (AA, AB, AN, and DHAA), and trichomes populations were therefore analysed in 3 types of leaves, in floral buds and flowers, and in three developmental stages: vegetative, floral budding and full flower. Although the maximum production of AN occurs when flowers are fully emerged, expression levels in the leaves of early pathway genes, HMGR, PFS, DXS, and DXR did not show close correlation with either AN or its precursors. However, later pathway genes, ADS and CYP, did correlate well with AN's immediate precursor, DHAA, in all leaf tissues tested. A close correlation between AN levels and leaf trichoma populations (as trichomes mm⁻2) was also observed (Arsenault et al., 2010a, manuscript submitted, submitted for publication).

DMSO Helps Elucidate a Possible ROS Role of DHAA in AN Biosynthesis:

Prior work showed that dimethyl sulfoxide (DMSO) increased artemisinin in A. annua seedling shoots (Towler and Weathers, 2007, Plant Cell Rep 26:2129-2136), but the mechanism of this serendipitous response was not understood. Interestingly it was the roots that were key to this DMSO response; artemisinin levels were not increased when only shoots of either rooted or unrooted shoots were treated with DMSO. This is not surprising, however, because the roots of A. annua are reported to play an important, but not as yet understood role in the production of artemisinin in the shoots (Ferreira and Janick, 1996, Plant Cell Tiss Organ Cult 44:211-217). Indeed rooted shoots of A. annua produce about 8 times the artemisinin of unrooted shoots, and in rooted shoots DMSO doubles that amount (Mannan et al., 2009, Plant Cell Rep, accepted for publication). In contrast, unrooted shoots are not responsive to DMSO. To determine if there is an optimum DMSO response, both the concentration of DMSO and duration of exposure were examined. At concentrations of DMSO between 0 and 2%, rooted shoots exhibited biphasic artemisinin production compared to the untreated controls with 2 peaks at 0.25 and 2% DMSO, both at about 2.26 times that of the control. At 0.5% DMSO, however, artemisinin production significantly decreased relative to the production at the peaks. Using the 0.25% DMSO concentration peak to determine the kinetics of the effect, we determined that the production of AN along with its precursor, DHAA, persisted for 7 days (Mannan et al., 2009, Plant Cell Rep, accepted for publication).

To investigate this DSMO response further, real time PCR was used to measure the transcriptional response of the artemisinic pathway genes, ADS and CYP, in both the shoot and root tissues of A. annua rooted shoot cultures after incubation in DMSO. The first gene in the artemisinin biosynthetic pathway, ADS, showed no significant increase in transcript level in response to DMSO compared to controls. On the other hand, the second gene in the pathway, CYP, did respond to DMSO but at a level of transcripts inverse to the amount of artemisinin (Mannan et al., 2009, Plant Cell Rep, accepted for publication). These results suggested that DMSO may be altering artemisinin production in some other way.

DMSO can act as both a reducing and an oxidizing agent, and can also associate with unshared pairs of electrons in the oxygen of alcohols, and may even act as a “radical trap” whereby as an intermediate in radical transfer, it may promote peroxidation (Kharasch and Thyagarajan 1983, Annal NY Acad Sci 411:391-402). Weathers et al. (1999) had previously shown that in a highly oxygenated environment more artemisinin is produced than in a hypoxic one. Wallaart et al. (1999, J Nat Prod 62:430-433; 2001, Planta 212:460-465) had suggested earlier that DHAA may be acting as a reactive oxygen species (ROS) scavenger and indeed the DMSO data are consistent with the hypothesis that the DMSO-induced ROS were possibly causing the increase in production of DHAA and, thus, providing the extra oxygens needed for the final biosynthetic step leading to AN.

To explore this further, rooted shoots were incubated in increasing DMSO concentrations and then stained with 3,3′-diaminobenzidine-HCl (DAB), which is specific for the specific ROS, H₂O₂. Although the increasing DMSO concentrations did not affect growth, the level of DAB staining in the leaves of rooted plantlets showed an increased in situ production of H₂O₂ in the foliage. In contrast, unrooted shoots showed no ROS formation in the presence of DMSO; roots were required for the ROS response in the shoots (Mannan et al., 2009, Plant Cell Rep, accepted for publication).

If DMSO was indeed increasing ROS production in the leaves of A. annua plantlets, then a natural ROS scavenger like ascorbic acid should inhibit both ROS and AN production. DMSO-induced hydrogen peroxide levels and artemisinin levels were both inhibited by addition of ascorbate. Together these data show that at least in response to DMSO, artemisinin production and hydrogen peroxide increase, and that when in situ hydrogen peroxide is reduced, so also is artemisinin suggesting that the ROS, hydrogen peroxide, may play a role in artemisinin production in A. annua.

Sugar Metabolism May also Play a Role in Regulating Artemisinin Biosynthesis:

In A. annua seedlings, glucose in particular was shown to stimulate artemisinin production (Wang and Weathers, 2007). Indeed it is the ratio of glucose to fructose that is important in regulating AN production. When seedlings were grown in sucrose-free medium, increasing artemisinin levels were directly proportional to increasing glucose as the ratio of glucose to fructose was increased from 0 to 100%. In comparison to sucrose or glucose, fructose inhibits the production of artemisinin. Other primary and secondary metabolites have been shown to be sugar responsive including products of the glyoxylate cycle (Graham et al., 1994, Plant Cell 6: 761-772) and anthocyanins (Vitrac et al. 2000, Phytochem. 53:659-665). Although in both Vitis and Arabidopsis, a number of anthocyanin genes have been shown to be unregulated in response to sucrose (Gallop et al., 2001, Plant Sci 161:579-588, 2002, J Experimental Bot 53:1397-1409; Solfanelli et al., 2006, Plant Physiol. 140:634-646), the mechanism of action is not entirely known.

Using Artemisia annua seedlings, artemisinic metabolites and gene transcript responses were measured (Arsenault et al. 2010b, manuscript submitted, submitted, for publication) after growth for 0-14 d on sucrose, glucose, or fructose. The 6 genes measured by real time RT-PCR were: HMGR, FPS, DXS, DXR, ADS, and CYP. Compared to seedlings grown in sucrose, HMGR, FPS, DXS, DXR, ADS and CYP transcript levels increased in varied amounts and with varied kinetics after growth in glucose, but not in fructose. The kinetics of these transcripts over 14 days, however, was very different both in timing and intensity of response (Arsenault et al., 2010b, manuscript submitted, submitted for publication).

Using LC/MS intracellular concentrations of AH, DHAA, AA, and AB were also measured in response to the three sugars. Compared to sucrose-fed seedlings, AN levels were significantly increased in seedlings fed glucose, but inhibited in fructose-fed seedlings. In contrast, AB levels doubled in seedlings grown in fructose compared to those grown in glucose. The level of mRNA transcripts of many of the genes analyzed was often negatively correlated with the observed metabolite concentrations.

AN is a known phytotoxin, even against A. annua (Duke et al. 1987, Weed Sci 35:499-505), suggesting that it may also inhibit its own synthesis in planta. When seedlings were gown in increasing levels of AN, root elongation was inhibited and, interestingly, levels of AA fell to below detectable limits (Arsenault et al., 2010b, manuscript submitted, submitted for publication). Together these results show there is a complex interplay between exogenous sugars and early developmental cues on the biosynthesis of artemisinin and its precursor metabolites. The results also suggest that the dynamics of shifting sugar-concentrations in the plant also play a role in the in situ control of artemisinin metabolism.

A. annua as a Delivery System for Artemisin—Mouse Studies:

A. annua has a rich ethnopharmacological history in the Chinese Materia Medica as a therapeutic tea, and the plant, although not highly palatable, also has been used as a condiment by various Asian cultures (http://pfaf.org/database/plants.php!Artemisia+annua). While use of the tea is no longer recommended due to emergence of resistance, to our knowledge there has been no investigation of the use of A. annua plant material to treat patients. Considering that some plant secondary metabolites appear to have a more synergistic effect when provided in planta than in a purified form (Gilbert and Alves, 2003, Curr Medicin Chem 10:13-20), eating A. annua via a compacted capsule in combination with an ACT partner, may offer an alternative, safe, inexpensive mode of drug delivery. Towards that goal it was necessary to also show that artemisinin could actually move from ingested plant material in the gut into the bloodstream.

Artemisia annua L. seeds from a Chinese strain (PEG01; a gift to PJW from C Z Liu (Chinese Academy of Sciences) were germinated in soil and then transplanted to small (3 in×3 in×2.5 in deep) pots and grown in a growth chamber at 25° C. under full spectrum fluorescent lights at ˜90 μmol m⁻² sec⁻¹ with a 16 hr pbotoperiod to inhibit flowering. Plant material was harvested, dried and leaves stripped from stems.

To determine the bioavailability of artemisinin in mice from oral ingestion of A. annua plant material A. annua leaves were dried at room temperature and then pulverized into a homogenous mixture that was aliquoted both for assay to determine the level of artemisinin and to use as feed. Ground leaf samples were suspended in water, pelletized, and then fed once via orogastric gavage to anesthetized BL6xICR mice to insure quantitative ingestion of the plant material. Prior to feeding, mice were fasted for 24 hours with water given ad libitum and prior to gastric intubation. Mice were fed one of the following at a volume ≦0.4 mL per mouse: pelletized A. annua plant material containing 30.7 μg AN in toto, or pure artemisinin mixed into pelleted feed at either 30.7 or 1,400 μg per mouse. Animals were then anesthetized and exsanguinated in groups of three at 30 min, and 60 min post feeding. At the end of the study, gross pathological examination of animals' digestive system was performed to ensure that animals suffered no internal damage.

Artemisinin and related metabolic constituents were extracted from plant material and from mouse blood using toluene and petroleum ether, respectively. Samples from each were subsequently dried and resuspended in ethyl acetate before injection onto a GC-MS. GC separation was achieved using a DB-5MS column (30 m×25 mm×0.25 um) and a temperature gradient programmed at 2° C./min from 120° C. to 160° C. and held at 160° C. for 10 minutes and then heated to 300° C. at 10 deg/min. All heated zones (injector and detector) were maintained at 200° C. MS scans from 50-400 m/z and EI with 70 eV. Artemisinin was detected and quantified via total ion count and retention time based on a genuine external standard and corrected via an internal standard.

Artemisinin from Ingested Artemisia annua Leaves Passes Readily into the Bloodstream of Mice.

To our knowledge, there has been no bioavailability study of artemisinin from oral ingestion of A. annua leaves. One of the key concerns is the relative bioavailability of artemisinin to a patient from a drug that is administered in planta. Mice were used in this study to determine how much artemisinin, if any, would move from the plant material in the gut into the bloodstream.

The pharmacokinetics of AN administered to mice as either dried A. annua leaves or pure compound mixed with mouse chow were compared. In this preliminary study, measurements were only taken up to 60 min after feeding. However, some general conclusions can be drawn. When ˜31 μg pure AN was fed, no AN was detectable in blood up to 60 min. Upon feeding 1400 μg AN, the levels in the blood rose to 0.074 mg L⁻¹ after 60 min. On the other hand, feeding A. annua leaves equivalent to 31 μg AN led to a Cₘₐₓ of 0.087 mg L⁻¹ at a tₘₐₓ of 30 minutes. These results are similar to those of Rath et al. (2004), Am J Trop Med Hyg 70:128-132, who compared the pharmacokinetics in humans of AN delivered as a tea, to pure AN. The tea showed a tₘₐₓ of 30 minutes and the pure compound a tₘₐₓ of 2.3 h, consistent with our mouse data measured up to 60 minutes.

Of particular interest is the comparatively high level of transfer of artemisinin into the bloodstream from the plant material vs. the pure drug. There was 45 times more pure artemisinin fed to the mice than the amount fed via A. annua leaves, yet almost the same amount of AN appeared in the bloodstream. Furthermore, when equal amounts of pure drug and plant delivered drug (˜31 μg) were fed to each mouse, the amount of artemisinin found in the blood from the plant-fed material (˜87 μg L⁻¹ blood) far surpassed the level from delivered pure drug (undetectable). Taken together these results show that compared to the pure drug, the bioavailability of AN from dried plant material is apparently greater (Table 1). These results suggest that an alternative mode of delivery of artemisinin is possible.

Bioavailability of artemisinin after oral intake is crucial for assessing the potential of using an edible botanical drug. Equally important are pharmacokinetic studies to insure proper formulation of the drug dose to fee delivered from plants to patient. Current oral bioavailability data on artemisinin are mainly based on studies with artemisinin capsules or tea prepared from A. annua leaves. For example, Rath et al. (2004), Am J Trop Med Hyg 70:128-132, measured artemisinin plasma concentrations in healthy male volunteers after oral ingestion of either traditionally prepared A. annua tea or in solid form. Although the intake as tea showed a faster absorption than the solid form, there was no difference in bioavailability (Table 1; Rath et al, 2004, Am J Trop Med Hyg 70:128-132). On the other hand, bioavailability after oral intake was reported at 32% of the drug administered via an intramuscular route (Titulaer et al. 1990, J Pharm Pharmacol 42:810-813). Pharmacokinetic studies done with healthy male volunteers showed artemisinin has an absorption lag-time of 0.5-2 hours, with peak plasma concentrations at 1-3 hours post-administration and a relatively short half-life of 1-3 hours (Alin et al. 1996, Trans R Soc Trop Med Hyg 90:61-65, Ashton et al. 1998, Drug Metab Dispos 26:25-27, Titulaer et al. 1990, J Pharm Pharmacol 42:810-813).

Synergistic and Broader Effects of an in planta Delivered Drug:

Artemisinin may have a more synergistic effect when provided in planta than as a pure drug. Inhibition of human cytochrome P450s by herbal extracts of numerous species, including a number of traditional medicinal plants, has been extensively studied (Rodeiro et al., 2009, Phytother Res 23:279-282), thereby increasing serum half-life. Indeed Liu et al. (1992), Plant Cell Rep 11:637-640, showed that although several methoxylated flavonoids, e.g. chrysosplenol-D, isolated from A. annua leaves had no direct effect on P. falciparum, when combined with pure AN, there was a significant enhancement of AN activity that could only be attributed to the presence of these compounds. A number of these constitutive flavonoids are present at all stage of A. annua's growth (Baraldi et al., 2008, Biochem System Ecol 36:340-348), and also show some antimalarial activity, albeit at levels that are orders of magnitude less than AN (Willcox, 2009, J Alternat Complement Med 15:101-109). Chrysosplenetin, casticin, eupatin, and chrysosplenol-D appear to help activate AN in its interaction with hemin (Bilia et al., 2002, Life Sci 70:769-778, 2006, Phytomed 13:487-493). Thus, these in planta constituents in A. annua likely enhance the overall activity of the drug. Another possible benefit to ingestion of whole leaf material is that there may be less chance of resistance occurring because there is a combination of active agents acting in concert to attack the pathogen. Eating A. annua combined with an ACT partner, may, therefore, offer an alternative, safe, inexpensive mode of drug delivery via a compacted capsule. Indeed should future studies prove successful in patients (clinical trials are still needed), this approach may also eventually prove more useful than purified compounds for production and delivery of other drugs produced in edible plants that survive the digestive tract in planta. Likewise, use of this plant may also prove useful in treating a variety of other diseases and parasitic ailments.

To study oral delivery, we used data from Rath et al. (2004), Am J Trop Med Hyg 70:128-132, where AN was administered as a tea. From 5 g DW of A. annua leaves (>1% DW AN), 57.5 mg of AN were measured and provided to humans and 240 μg L⁻¹ appeared in the bloodstream. The minimum effective concentration of AN in the blood is ˜10 μg L⁻¹ (Alin and Bjorkman, 1994, Am J Trop Med Hyg 50:771-776). An adult human male weighing 70 kg has about 5 L of blood. The AN in a tea-extract from 5 g of dried leaves containing 1% (w/w) AN, therefore, provides considerably more AN (240 μg L⁻¹) than the minimum required in the blood suggesting that 1 g of ingested dried leaves could be more than adequate to deliver a single dose of AN to an adult patient.

As another comparison, mice have about 1.4 mL blood, while a 70 kg human male has about 5 L. Our mice were fed about 31 μg of AN and contained an average total of 0.12 μg AN in their blood, so to obtain the necessary total amount of AN in human blood, 50 μg are needed (10 μg AN mL⁻¹ is considered therapeutic) for a single AN dose. Assuming similar uptake, a patient would have to ingest 17 mg of AN from plant leaves. Assuming also a 1% AN content, which is possible to consistently obtain from some A. annua strains (e.g. the Artemis strain has ˜1.4%; Ferreira et al, 2005, Plant Genet Resources 3:206-229), 1-2 g of dried leaves would be adequate and reasonable to deliver a single dose of the drug to a 70 kg adult. For children smaller amounts would be required, which is easily accomplished using smaller capsules.

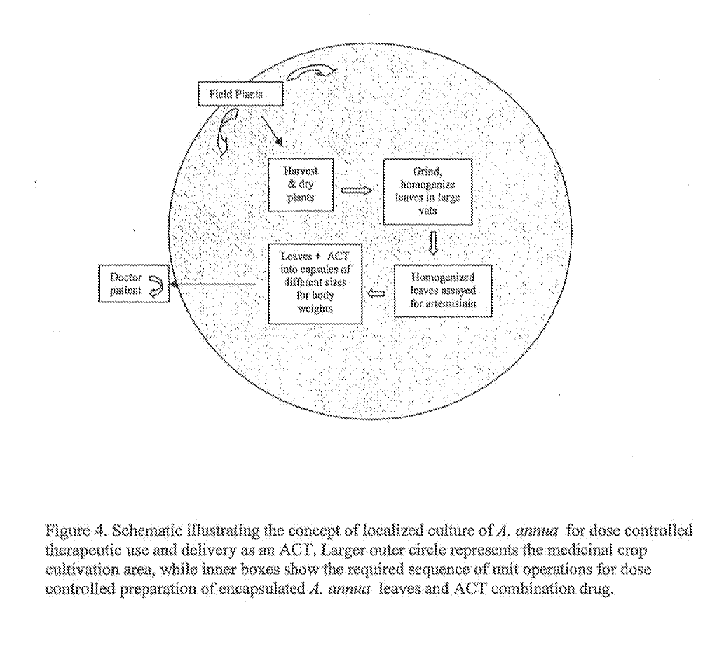

To provide a controlled delivery of the drug via oral delivery of dried plant material, plants must be harvested, dried, powdered, homogenized, and pooled into large containers where they can be assayed for artemisinin content using strategies that are easy, low cost, and quantitative (Widmer et al., 2007, J Alternat Complement Med 15:101-109: Koobkokkruad et al., 2007, L. Phtochem Anal 18:229-234). Capsules would then be loaded with compacted leaf powder of a known dosage to which the ACT drug partner can be added. Alternatively the ACT drug partner could be administered separately. This processing strategy (FIG. 4) is inexpensive, and also reliable for preparing known doses of artemisinin as dried plant material. If this processing facility were centered within a region where local farmers are growing the plant, the entire process could be self-sustaining thereby not only strengthening local health, but also the local economy.

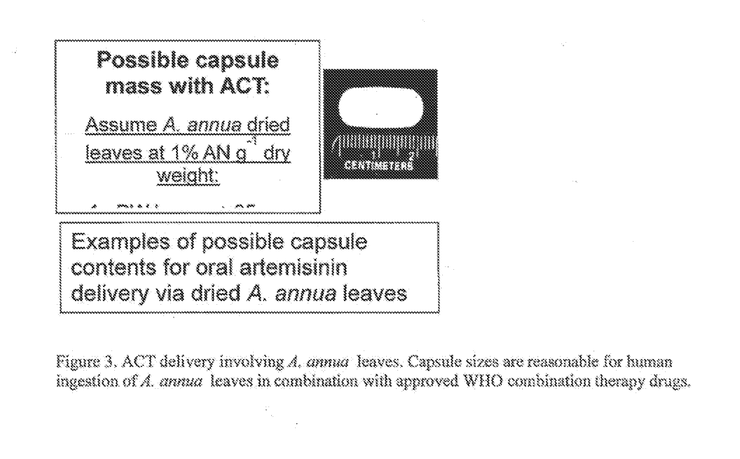

Although our data compare favorably with studies in rats and A. annua teas (Table 1), use of a tea is a monotherapy, involves no ACT, and is thus, counter-indicated by the WHO in an effort to minimize emergence of resistant strains of the pathogen. In contrast, our drug delivery plan would also incorporate an ACT drug partner as follows: plants are harvested and dried (WHO, 2006, http://www.who.int/medicines/publications/traditional/ArtemisiaMonograph.pdf); leaves are pulverized and homogenized in large vessels; samples are then taken to measure AN content to ensure preparation of adequate and controlled doses for patients; the assayed leaves are then compacted into capsules into which appropriate amounts of ACT partner drugs are added. As an example see FIG. 3. The caplet shown is about 1,300 mg so if the assayed dry leaves only contained 1% AN, about 1-2 capsules would need to be ingested per dose to treat a 70 kg human. WHO guidelines for human treatment specify additional AN doses throughout the day.

We therefore, submit that oral delivery of artemisinin via dried A. annua plant material and in conjunction with an ACT drug partner could provide an effective, low cost therapy for treating malaria and the other conditions set out above in developing countries. Despite the prevalence and preference of the modem medical community for single-ingredient-drugs, there are examples that illustrate the often ignored benefits of using complex botanical drugs vs. pure ones (Raskin et al. 2002, Trends Biotechnol 20:522-531). With the potential for synergistic benefits, drug delivery via natural sources may be preferable to that in an isolated form (Raskin et al., 2002, Trends Biotechnol 20:522-531; Gilbert and Alves, 2003, Curr Medicin Chem 10:13-20). We have shown that when provided directly from plant material, high levels of artemisinin can be detected in the bloodstream of mice. We further proposed a simple method for insuring a controlled dose of artemisinin via in planta delivery that when combined with the simple methods for stimulating increases of the drug while the crop is in the field, may provide significant relief to the shortage of low cost artemisinin available for use to treat malaria and other neglected diseases in developing countries.

TABLE 1
Comparison of maximum artemisinin detected in serum or plasma from orally
ingested pure artemisinin, whole plant A. annua, or prepared tea.

| | | | | Plasma/serum | |
|---|---|---|---|---|---|
| | | | | concentration | |
| | Dose per | | | Artemisinin | |
| Drug delivery form | individual | | Subject | Cₘₐₓ (mg L⁻¹) | Reference |
| Pure artemisinin | 500 | mg | Healthy | 0.6 | Dien et al. |
| | | | human males | | (1997) |
| | 500 | mg | | 0.3 | de Vries et al. |
| | | | | | (1997) |
| Tea extract: | 57.5 | mg | Healthy | 0.2 | Rath et al. |
| 5 g dried leaves | | | human males | | (2004) |
| Pure artemisinin control | 500 | mg | | 0.5 | |
| Intragastric delivery is 1:9 | 10 | mg kg⁻¹ | Rats | 0.8 | Li et al. |
| dimethyl-acetamide-oil^{a} | ^{b}2,320 | μg rat⁻¹ | | | (1998) |
| Whole plant: | 30.7 | μg mouse⁻¹ | Mice | 0.087 | This study. |
| Dried leaves | | | | | |
| Pure artemisinin control | 30.7 | μg mouse⁻¹ | | Not detectable. | |
| | 1400 | μg mouse⁻¹ | | ≧0.074 | |

^{a}Delivered as dihydroartemisinin.
^{b}Rat body weights ranged from 210-254 g; we used 232 as an average to calculate total μg delivered to each animal.

### Compositions

The compositions described herein include A. annua tissue and a pharmaceutically acceptable carrier. Artemisia annua L. is also known by vernacular names, including, for example, annual wormwood or sweet wormwood in English; Caohao, Cao Qinghao, Cao Haozi, Chouhao, Chou Qinghao, Haozi, Jiu Bingcao, Kuhao, San Gengcao, Xianghao, Xiang Qinghao, Xiang Sicao, Xiyehao in Chinese; armoise annuelle in French; Kusoninjin in Japanese; Chui-ho, Hwang-hwa-ho, Gae-tong-sook in Korean; and Thanh cao hoa vàng in Vietnamese. Any cultivated or wild variety of A. annua can be used. Methods of cultivating A. annua are well known in the art; exemplary methods are described in the WHO Monograph on Good Agricultural and Collection Practices [GACP] for Artemisia annua L. (2006), which is herein incorporated by reference.

Any artemesinin-containing tissue can be used. Artemesinin is generally produced in glandular trichomes found on leaves, floral buds and flowers. The artemisinin content of A. annua harvested from different production areas and different cultivation conditions can vary widely. The content of artemisinin is affected by numerous factors such as geographical conditions, harvesting time, temperature and fertilizer application. Harvesting at the appropriate time is important to ensure optimum content of artemisinin in A. annua. Harvest time should be determined by a study of the weather conditions, dynamic accumulation and local harvesting experience. The yield of A. annua leaves and the content of artemisinin may be reduced if harvesting is either too early or is delayed. For some locations, the ideal harvesting time is the early stage of flower budding. The content of artemisinin of A. annua can be tested before harvesting. Methods for assaying artemisinin content are well known in the art and include, for example, extraction of the leaves with organic solvents followed by thin layer chromatograhy or by HPLC. The content of artemesinin can vary but the peak content can range from about 0.5% to about 3.0%, e.g., from about 0.5% to about 2.5%, from about 1% to about 2.0% dry weight of the leaves of A. annua.

The A. annua tissue can be harvested by hand using, for example, machetes, shears, saws or by mechanicanized methods. The crop can be cut down and optionally processed before drying. The processing steps may involve one or more of removal of foreign matter, e.g., insects, dirt, non-A. annua plant tissue, rinsing or spraying with water, brief softening in water, cutting the A. annua tissue into smaller pieces, stripping specific tissue types from the plant, e.g., leaves, floral buds or flowers and segregating them processing independently. Any standard drying method can be used including, for example, sun-drying, shade-drying and oven-drying.

For ease of handling and administration, the dried A. annua tissue can be compacted. For example the dried tissue may crushed, shredded, cut, granulated, pulverized, ground or powdered, using art-known methods.

The A. annua tissues described herein may include, in addition to artemisinin, a wide variety of compounds that may also provide therapeutic benefits, for example artemisinin I, artemisinin II, artemisinin III, artemisinin IV, artemisinin V, artemisic acid, artemisilactone, artemisinol, epoxyarteannuinic acid, artemisia ketone, 1,8-cineole, camphene hydrate, cuminal, sesquiterpenoids, flavonoids (e.g., artemetin, casticin, chrysoplenetin, chrysosplenol-D and cirsilineol), coumarins, proteins (such as β-galactosidase, β-glucosidase), and steroids (e.g. β-sitosterol and stigmasterol).

### Pharmaceutical Carriers

The compositions also include a pharmaceutically acceptable carrier. We use the terms “pharmaceutically acceptable” (or “pharmacologically acceptable”) to refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

This invention also includes pharmaceutical compositions which contain, as the active ingredient, the A. annua tissues described herein, in combination with one or more pharmaceutically acceptable carriers. In some embodiments, the A. annua tissue can be sterilized using conventional sterilization techniques before or after it is combined with the pharmaceutically acceptable carrier, in making the compositions of the invention, the A. annua is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. As is known in the art, the type of diluent can vary depending upon the intended route of administration. The resulting compositions can include additional agents, such as preservatives. The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The pharmaceutical compositions can be formulated so as to provide quick, sustained or delayed release of the active ingredient after administration to the patient by employing procedures known in the art.

Pharmaceutically acceptable compositions for use in the present methods, including those in which A. annua tissue is entrapped in a colloid for oral delivery, can be prepared according to standard techniques. The A. annua tissue can be dried and compacted by grinding or pulverizing as described above and the compacted tissue inserted into a capsule for oral administration. In some embodiments, the A. annua tissue can be combined one or more excipients, for example, a disintegrant, a filler, a glidant, or a preservative. Suitable capsules include both hard shell capsules or soft-shelled capsules. Any lipid-based or polymer-based colloid may be used to form the capsule. Exemplary polymers useful for colloid preparations include gelatin, plant polysaccharides or their derivatives such as carrageenans and modified forms of starch and cellulose, e.g., hypromellose. Optionally, other ingredients may be added to the gelling agent solution, for example plasticizers such as glycerin and/or sorbitol to decrease the capsule's hardness, coloring agents, preservatives, disintegrants, lubricants and surface treatment. In some embodiments, the capusule does not include gelatin. In other embodiments, the capsule does not include plant polysaccharides or their derivatives.

Regardless of their original source or the manner in which they are obtained, the A. annua tissues of the invention can be formulated in accordance with their use. These compositions can be prepared in a manner well known in the pharmaceutical art, and can be administered by a variety of routes, depending upon whether local or systemic treatment is desired and upon the area to be treated. Administration may be oral or topical (including ophthalmic and to mucous membranes including intranasal, vaginal and metal delivery). In some embodiments, administration can be pulmonary (e.g., by inhalation, or insufflation of powders or aerosols, including by nebulizer; intratracheal, intranasal, epidermal and transdermal) or ocular. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

The compositions can be formulated in a unit dosage form, each dosage containing, for example, from about 0.1 gram to about 5.0 grams, from about 0.2 gram to about 4.5 grams, from about 0.3 gram to about 4.0 grams, from about 0.4 gram to about 3.5 grams, from about 0.5 gram to about 3.0 grams, from about 0.6 gram to about 2.5 grams, from about 1.0 gram to about 2.0 grams of A. annua tissue.

The term “unit dosage forms” refers to physically discrete units suitable as unitary dosages for human subjects and other mammals, each unit containing a predetermined quantity of active material calculated to produce the desired, therapeutic effect, in association with a suitable pharmaceutical excipient. For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition, so that the composition can be readily subdivided into equally effective unit dosage forms such as tablets, pills and capsules. This solid preformulation is then subdivided into unit dosage forms of the type described above containing from, for example, 1.0 gram to about 2.0 grams of the A. annua tissue of the present invention.

In some embodiments, tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

The proportion or concentration of the compounds of the invention in a pharmaceutical composition can vary depending upon a number of factors including dosage, chemical characteristics (e.g., hydrophobicity), and the route of administration. For example, the A. annua tissues of the invention can be provided in a capsule containing from about 1 gram to about 2 grams of tissue for oral administration.

### Methods of Treatment

The compounds disclosed herein are generally and variously useful for treatment of infectious diseases, e.g., a parasitic disease, inflammatory diseases and cancer. A patient is effectively treated whenever a clinically beneficial result ensues. This may mean, for example, a complete resolution of the symptoms of a disease, a decrease in the severity of the symptoms of the disease, or a slowing of the disease's progression. In the case of an infectious disease, an effective treatment may mean the elimination of all or substantially all of the infectious agent from the patient's body. These methods can further include the steps of a) identifying a subject (e.g., a patient and, more specifically, a human patient) who has an infectious disease, e.g., a parasitic disease, inflammatory disease or cancer; and b) providing to the subject a composition described herein. An amount of such a compound provided to the subject that results in a complete resolution of the symptoms of a disease, a decrease in the severity of the symptoms of the disease, or a slowing of the disease's progression is considered a therapeutically effective amount. The present methods may also include a monitoring step to help optimize dosing and scheduling as well as predict outcome. For example, monitoring can be used to detect the onset of drug resistance and to rapidly distinguish responsive patients from unresponsive patients. Where there are signs of resistance or nonresponsiveness, a physician can choose an alternative or adjunctive agent before the disease develops additional escape mechanisms.

Patients amenable to treatment include patients with a parasitic disease, for example malaria, schistosomiasis, clonorchiasis and other trematode infections including Schistosoma japonicum, S. mansoni, S. haematobium, Clonorchis sinensis, Fasciola hepatica and Opisthorchis viverrini. The malaria can be due to any species of plasmodium including, for example, Plasmodium falciparum, Plasmodium vivax, Plasmodium ovale, Plasmodium Malariae, Plasmodium knowlesi, P. inui, P. cynomolgi, P. simiovale, P. brazilianum, P. schwetzi and P. simium

Patients amenable to treatment include patients with any of a wide variety of cancers or neoplastic disorders, including, for example, without limitation, breast cancer, hematological cancers such as myeloma, leukemia and lymphoma (e.g., Burkitt lymphoma, non-Hodgkin lymphoma, Hodgkin lymphoma, and acute T cell leukemia) neurological tumors such as brain tumors, e.g., gliomas, including astrocytomas or glioblastomas, melanomas, lung cancer, head and neck cancer, thyroid cancer, gastrointestinal tumors such as stomach, colon or rectal cancer, liver cancer, pancreatic cancer, genitourinary tumors such ovarian cancer, vaginal cancer, vulval cancer, endometrial cancer, bladder cancer, kidney cancer, testicular cancer, prostate cancer, or penile cancer, bone tumors, vascular tumors, and skin cancers such as basal cell carcinoma, squamous cell carcinoma and melanoma.

The methods disclosed herein can be applied to a wide range of species, e.g., humans, non-human primates (e.g., monkeys), horses or other livestock, dogs, cats or other mammals kept as pets, rats, mice, or other laboratory animals. The compounds described herein are useful in therapeutic compositions and regimens or for the manufacture of a medicament for use in treatment of diseases or conditions as described herein (e.g., a parasitic infection or a cancer disclosed herein).

The therapeutic dosage of the compounds of the present invention can vary according to, for example, the particular use for which the treatment is made, the manner of administration of the compound, the health and condition of the patient, and the judgment of the attending clinician. The proportion or concentration of a compound of the invention in a pharmaceutical composition can vary depending upon a number of factors including dosage, chemical characteristics (e.g., hydrophobicity), and the route of administration. For example, the A. annua tissues of the invention can be provided in a capsule containing about 1.0 to about 2.0 grams of the compound for parenteral administration.

Any of the compositions described herein can be formulated such that the tissue is in a unit dosage form of about 0.1 grams to about 5.0 grams (e.g., about 0.1, 0.2, 0.5, 1.0, 1.5, 2.0, 2.5, 3.0, 3.5, or 40 grams; with ranges being selected from between any lower and higher level (e.g., about 0.5-1.0 gram, 1.0-2.0 grams, or about 2.5-3.0 grams).

The dosage may also be expressed as the amount that gives rise to a circulating blood or plasma level. For example, a unit dosage form can include an amount of Artemisia annua sufficient, when administered to a subject, to result in a circulating concentration of artemesinin in the subject of more than 0.2 mg/L (e.g., about 0.3 mg/L to about 1.0 mg/L (e.g., about 0.4, 0.5, 0.6, 0.7, or 0.8 mg/L)). The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by oral or topical administration. In some embodiments, the compositions can be administered by intravenous, intracranial, intraperitoneal, intramuscular, subcutaneous, intramuscular, intrarectal, intravaginal, intrathecal, intratracheal, intradermal, or transdermal injection, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Any method known to those in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

The compounds described herein may also be administered with another therapeutic agent, such as a standard antiparasitic agent, cytotoxic agent, or cancer chemotherapeutic. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks. In some embodiments, the A. annua tissue and the pharmaceutical carrier may be combined with the standard agent in a single formulation. Exemplary antiparasitic agents include, without limitation, lumefantrine, mefloquine, amodiaquine or sulfadoxine/pyrimethamine.

The compositions may also be administered along with a conventional cancer treatment, e.g., radiotherapy, chemotherapy, a biologic agent or surgical intervention. The pharmaceutical compositions can also include antibodies, e.g., antibodies that recognize additional cellular targets. Exemplary immunoglobulins are listed below. Each immunoglobulin is identified by its proper name and its trade name. Numbers in parenthesis beginning with “DB” refer to the identifiers for each antibody on The DrugBank database available at the University of Alberta. The DrugBank database is described in Wishart D S, Knox C, Guo A C, et al. (2008). “DrugBank: a knowledgebase for drugs, drug actions and drug targets”. Nucleic Acids Res. 36 (Database issue): D901-6 and can be accessed at www.drugbank.ca. Useful immunoglobulins include: Abciximab (ReoPro™) (DB00054), the Fab fragment of the chimeric human-murine monoclonal antibody 7E3, the synthesis of which is described in EP0418316 (A1) and WO8911538 (A1), which are herein incorporated by reference; Adalimumab (Humira™) (DB00051), a fully human monoclonal antibody that binds to Tumor Necrosis Factor alpha (TNF-.alpha.) and blocks TNF-.alpha. binding to its cognate receptor; alemtuzumab (Campath™) (DB00087), a humanized monoclonal antibody that targets CD52, a protein present on the surface of mature lymphocytes, used in the treatment of chronic lymphocytic leukemia (CLL), cutaneous T cell lymphoma (CTCL) and T-cell lymphoma; basiliximab (Simulect™) (DB00074), a chimeric mouse-human monoclonal antibody to the .alpha. chain (CD25) of the IL-2 receptor; bevacizumab (Avastin™) (DB00112) a humanized monoclonal antibody that recognises and blocks vascular endothelial growth factor (VEGF), the chemical signal that stimulates angiogenesis, the synthesis of which is described in Presta L G, Chen H, O'Connor S J, et al Humanization of an anti-vascular endothelial growth factor monoclonal antibody for the therapy of solid tumors and other disorders. Cancer Res, 57: 4593-9, 1997; certuximab (Erbitux™) (DB00002), a chimeric (mouse/human) monoclonal antibody that binds to and inhibits the epidermal growth factor receptor (EGFR), the synthesis of which is described in U.S. Pat. No. 6,217,866, which is herein incorporated by reference; certolizumab pegol (Cimzia™), a PEGylated Fab′ fragment of a humanized TNF inhibitor monoclonal antibody; daclizumab (Zenapax™) (DB00111), a humanized monoclonal antibody to the alpha subunit of the IL-2 receptor; eculizumab (Soliris™), a humanized monoclonal antibody that binds to the human C5 complement protein; efalizumab (Raptiva™) (DB00095), a humanized monoclonal antibody that binds to CD11a, gemtuzumab (Mylotarg™) (DB00056) a monoclonal antibody to CD33 linked to a cytotoxic agent, the amino acid sequence of which is described in J Immunol 148:1149, 1991) (Caron P C, Schwartz M A, Co M S, Queen C, Finn R D, Graham M C, Divgi C R, Larson S M, Scheinberg D A. Murine and humanized constructs of monoclonal antibody M195 (anti-CD33) for the therapy of acute myelogenous leukemia. Cancer. 1994 Feb. 1; 73 (3 Suppl):1049-56); ibritumomab tiuxetan (Zevalin™) (DB00078), a monoclonal mouse IgG1 antibody ibritumomab in conjunction with the chelator tiuxetan and a radioactive isotope (yttrium⁹⁰ or indium¹¹¹); Infliximab (Remicade™) (DB00065), a chimeric mouse-human monoclonal antibody that binds to tumour necrosis factor alpha (TNF.alpha.), the synthesis of which is described in U.S. Pat. No. 6,015,557, which is herein incorporated by reference; muromonab-CD3 (Orthoclone OKT3™), a mouse monoclonal IgG2a antibody that binds to the T cell receptor-CD3-complex; natalizumab (Tysabri™) (DB00108), a humanized monoclonal antibody against the cellular adhesion molecule .alpha.4-integrin, the sequence of which is described in Leger O J, Yednock T A, Tanner L, Horner H C, Hines D K, Keen S, Saldanha J, Jones S T, Fritz L C, Bendig M M. Humanization of a mouse antibody against human alpha-4 integrin: a potential therapeutic for the treatment of multiple sclerosis. Hum Antibodies. 1997; 8 (1):3-16; omalizumab (Xolair™) (DB00043), a humanized IgGlk monoclonal antibody that selectively binds to human immunoglobulin E (IgE); palivizumab (Synagis™) (DB00110), a humanized monoclonal antibody (IgG) directed against an epitope in the A antigenic site of the F protein of the Respiratory Syncytial Virus (RSV), the amino acid sequence of which is described in Johnson S, Oliver C, Prince G A, Hemming V G, Pfarr D S, Wang S C, Dormitzer M, O'Grady J, Koenig S, Tamura J K, Woods R, Bansal G, Couchenour D, Tsao E, Hall W C, Young J F. Development of a humanized monoclonal antibody (MEDI-493) with potent in vitro and in vivo activity against respiratory syncytial virus. J Infect Dis. 1997 November; 176 (5): 1215-24; panitumumab (Vectibix™), a fully human monoclonal antibody specific to the epidermal growth factor receptor (also known as EGF receptor, EGFR, ErbB-1 and HER1 in humans); ranibizumab (Lucentis™), an affinity matured anti-VEGF-A monoclonal antibody fragment derived from the same parent murine antibody as bevacizumab (Avastin); rituximab (Rituxan™, Mabthera™) (DB00073), a chimeric monoclonal antibody against the protein CD20, which is primarily found on the surface of B cells; tositumomab (Bexxar™) (DB00081), a anti-CD20 mouse monoclonal antibody covalently bound to .sup.131I; or trastuzumab (Herceptin™) (DB00072), a humanized monoclonal antibody that binds selectively to the HER2 protein.

The antibodies can include bioequivalents of the approved or marketed antibodies (biosimilars). A biosimilar can be for example, a presently known antibody having the same primary amino acid sequence as a marketed antibody, but may be made in different cell types or by different production, purification or formulation methods. Generally any deposited materials can be used.

The pharmaceutical compositions may also include or be administered along with a cytotoxic agent, e.g., a substance that inhibits or prevents the function of cells and/or causes destruction of cells. Exemplary cytotoxic agents include radioactive isotopes (e.g., ¹³¹I, ¹²⁵I, ⁹⁰Y and ¹⁸⁶Re), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein). Such agents may be conjugated, coupled, linked or associated with an antibody disclosed herein.

Conventional cancer medicaments can be administered with the compositions disclosed herein. Useful medicaments include anti-angiogenic agents, i.e., agents block the ability of tumors to stimulate new blood vessel growth necessary for their survival. Any anti-angiogenic agent known to those in the art can be used, including agents such as Bevacizumab (Avastin®, Genentech, Inc.) that block the function of vascular endothelial growth factor (VEGF). Other examples include, without limitation, Dalteparin (Fragmin®), Suramin ABT-510, Combretastatin A4 Phosphate, Lenalidomide, LY31761S (Enzastaurin), Soy Isoflavone (Genistein; Soy Protein Isolate) AMG-706, Anti-VEGF antibody, AZD2171, Bay 43-9006 (Sorafenib tosylate), PI-88, PTK787/ZK 222584 (Vatalanib), SU11248 (Sunitinib malate), VEGF-Trap, XL184, ZD6474, Thalidomide, ATN-161, EMD 121974 (Cilenigtide) and Celecoxib (Celebrex®).

Other useful therapeutics include those agents that promote DNA-damage, e.g., double stranded breaks in cellular DNA, in cancer cells. Any form of DNA-damaging agent know to those of skill in the art can be used. DNA damage can typically be produced by radiation therapy and/or chemotherapy. Examples of radiation therapy include, without limitation, external radiation therapy and internal radiation therapy (also called brachytherapy). Energy sources for external radiation therapy include x-rays, gamma rays and particle beams; energy sources used in internal radiation include radioactive iodine (iodine¹²⁵ or iodine¹³¹), and from strontium⁸⁹, or radioisotopes of phosphorous, palladium, cesium, iridium, phosphate, or cobalt. Methods of administering radiation therapy are well know to those of skill in the art.

Examples of DNA-damaging chemotherapeutic agents include, without limitation, Busulfan (Myleran), Carboplatin (Paraplatin), Carmustine (BCNU), Chlorambucil (Leukeran), Cisplatin (Platinol), Cyclophosphamide (Cytoxan, Neosar), Dacarbazine (DTIC-Dome), Ifosfamide (Ifex), Lomustine (CCNU), Mechlorethamine (nitrogen mustard, Mustargen), Melphalan (Alkeran), and Procarbazine (Matulane).

Other standard cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; folinic acid; purine analog antimetabolites, mercaptopurine; pyrimidine analog antimetabolites, such as fluorouracil (5-FU) and gemcitabine (Gemzar®); hormonal antineoplastics, such as goserelin, leuprolide, and tamoxifen; natural antineoplastics, such as aldesleukin, interleukin-2, docetaxel, etoposide (VP-16), interferon alfa, paclitaxel (Taxol®), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin®), Schizophyllan, cytarabine (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, carminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimex®), interferon-β, mepitiostane, mitobronitol, melphalan, laminin peptides, lentinan, Coriolus versicolor extract, tegafur/uracil, estramustine (estrogen/mechlorethamine).

Additional agents which may be used as therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β, and γ hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; and anti-angiogenesis factors.

### EXAMPLES

### Example 1

### Effect of Orogastic Administration of Artemisia annua Leaf Samples on Parasitemia in Plasmodium chabaudi-Infected Mice

We will compare the effects of administering Artemisia annua leaf samples and purified artemesinin on parasitemia in mice that have been infected with Plasmodium chabaudi.

P. chabaudi parasites are passaged in pathogen free mice by injection of 100 ul of 10⁵ pRBC/ml. Infected erythrocytes are collected ten days post-infection by retroorbital bleeding. Twenty four C57/B16 mice (6 to 8 weeks old) mice will be infected with 10⁵ infected erythrocytes. On day 2 after injection, the mice will be fasted for 24 hours with water given ad libitum. On day 3 after injection, the mice will be anesthetized using isoflurane and administered either ground Artemisia leaf samples (8 mice) or purified artemesinin (8 mice) via orogastric gavage. The remaining 8 mice will not receive either ground Artemisia leaf samples or purified artemesinin.

For Artemisia leaf sample preparation, Artemisia annua L. seeds from a Chinese strain (PEG01; a gift to PJW from C Z Liu (Chinese Academy of Sciences) will be germinated in soil and then transplanted to small (3 in×3 in×2.5 in deep) pots and grown in a growth chamber at 25° C. under full spectrum fluorescent lights at ˜90 μmol m⁻² sec⁻¹ with a 16 hr photoperiod to inhibit flowering. Plant material will be harvested, dried at room temperature and leaves stripped from stems. The leaves will then be pulverized into a homogenous mixture. The ground Artemisia leaf samples will be resuspended in water (60 milligrams in 1.0 ml). 0.4 ml of this solution will be administered via orogastric gavage per mouse. Purified artemesinin will be obtained from Novartis; 10 mg of artemisinin will be resuspended in 1 gram of powdered mouse food pellet and 10 ml of water. 0.4 ml of this solution will be administered via orogastric gavage per mouse. Parasitemia, as the percentage of infected erythrocytes, will be determined in tail-vein blood smears by counting 400 cells per smear and by FACS analysis. The parasitemia will be measured daily before treatment with Artemisia annua leaves or purified artemisinin, and 6, 12, 18, 24, 36, 72 hrs, up to 10 days post treatment for all mice. At day 15 the mice will be euthanized. The experiment will be repeated 3 times.

A number of embodiments of the invention have been described. Nevertheless, it will be understood that various modifications may be made without departing from the spirit and scope of the invention. Accordingly, other embodiments are within the scope of the following claims.

1. A pharmaceutical composition comprising Artemisia annua tissue and a pharmaceutically acceptable carrier.

2. The composition of claim 1, wherein the Artemisia annua tissue comprises leaves, floral buds, or flowers.

3. The composition of claim 1, wherein the tissue is harvested at a time when artemesinin levels in the tissue are between about 0.5-3.0% dry weight of the tissue.

4. The composition of claim 1, wherein the Artemisia annua tissue comprises shredded tissue, cut tissue, granulated tissue, pulverized tissue, ground tissue or powdered tissue.

5. (canceled)

6. The composition of claim 1, wherein the pharmaceutically acceptable carrier comprises a lipid-based or polymer-based colloid.

7-8. (canceled)

9. The composition of claim 1, wherein the tissue is in a unit dosage form of about 0.1 grams to about 5.0 grams.

10. (canceled)

11. The composition of claim 9, wherein the unit dosage form comprises an amount of Artemisia annua sufficient, when administered to a subject, to result in a circulating concentration of artemesinin in the subject within a range of about 0.3 mg/L to about 1.0 mg/L.

12. (canceled)

13. The composition of claim 1, further comprising an anti-parasitic agent.

14. The composition of claim 13, wherein the anti-parasitic agent comprises an anti-malarial agent.

15-16. (canceled)

17. A method of treating a subject who has a parasitic disease, the method comprising administering to the subject an effective amount of a composition comprising Artemisia annua tissue and a pharmaceutically acceptable carrier.

18. The method of claim 17, further comprising identifying a subject who has a parasitic disease.

19-21. (canceled)

22. The method of claim 17, wherein the Artemisia annua tissue comprises leaves, floral buds, or flowers.

23. The method of claim 17, wherein the tissue is harvested at a time when artemesinin levels in the tissue are between about 0.5-3.0% dry weight of the tissue.

24. The method of claim 17, wherein the Artemisia annua tissue comprises shredded tissue, cut tissue, granulated tissue, pulverized tissue, ground tissue or powdered tissue.

25. (canceled)

26. The method of claim 17, wherein the pharmaceutically acceptable carrier comprises a lipid-based or polymer-based colloid.

27-28. (canceled)

29. The method of claim 17, wherein the tissue is in a unit dosage form of about 0.1 grams to about 5.0 grams.

30. (canceled)

31. The method of claim 17, wherein the composition is administered for a time and in an amount sufficient to result in a circulating concentration of artemesinin in the subject within a range of about 0.3 mg/L to about 1.0 mg/L.

32. (canceled)

33. The method of claim 17, wherein the composition is formulated for oral administration.

34. The method of claim 17, further comprising administering an anti-parasitic agent.

35. The method of claim 34, wherein the anti-parasitic agent comprises an anti-malarial agent.

36.-37. (canceled)
